(19) United States
(12) Patent Application Publication
(10) Pub. No.: US 2012/0190057 A1
(43) Pub. Date: Jul. 26, 2012

(54) Method for the qualitative and/or quantitative analysis of tumour cells

(75) Inventors: Khatuna Elizbarowna Gvichiya, St. Petersburg (RU); Michael Edetsberger, Wien (AT); Martin Knapp, Wien (AT)

(73) Assignee: Onkotec GmbH, Waidhofen/Thaya (AT)

(21) Appl. No.: 13/321,817

(22) PCT Filed: May 19, 2010
(86) PCT No.: PCT/AT2010/000169

§ 371 Date: Apr. 9, 2012

(30) Foreign Application Priority Data
May 19, 2009 (AT) A 784/2009

(51) Int. Cl. G01N 21/64

(52) U.S. Cl. 435/29

(57) ABSTRACT

A method for qualitative and/or quantitative analysis of tumor cells, including the following steps: a) generating a dye complex from an aminocoumarin and cyclodextrin; b) mixing the tumor cells with the dye complex prepared in step a); c) incubating the cells with the dye complex prepared in step a); d) analyzing the tumor cells by means of fluorescence microscopy and/or fluorescence spectrometric analysis.

The invention relates to a method and means for marking tumor cells. In particular, the invention relates to a method and means for qualitative and qualitative analysis of tumor cells.

In dedifferentiated tumor cells, classical cytology functions quite well. Conversely, well-differentiated tumor cells can be distinguished from healthy cells only with great difficulty using classical cytological methods. Hence there is a need for methods and means which are capable of qualitative and/or quantitative analysis of well-differentiated tumor cells in the presence of normal cells, especially in body fluids.

There is no doubt that fluorescing organic substances will play an important role in future technologies, such as biosensors, photocatalysts, or optoelectronics, or already do so. Especially in the group of aminocoumarins, there are some that are already being used successfully in many fields because of their photophysical qualities, such as photostability and optimal quantum yield [1-4].

The use of aminocoumarin in nanoparticles is also known; however, the aminocoumarin serves solely for marking and characterizing the stability of nanoparticles, not for direct fluorescent marking of cells [5], since the fluorescence marking is dependent indirectly on the uptake of the nanoparticles. In the cells described, the dye serves the purpose of selective marking of the nanoparticles, and the takeup mechanisms and interactions between nanoparticles and tumor cells are being investigated.

The aminocoumarins are organic substances which in an aqueous environment have very low solubility or are virtually insoluble [6, 7].

However, it is known that coumarin 6 can be kept in solution both in an aqueous and a buffered environment by means of cyclodextrins (including cycloamyloses known as Schardinger dextrins, or cycloglucanes and cyclical oligosaccharides), since cyclodextrins, with other molecules of suitable size and polarity, can form inclusion complexes [7, 8, 13]. Especially the hydrophilic cyclodextrins are capable of varying the output rates and the timing profile of the release of substances [9]. Cyclodextrins have also been used very successfully as stabilizers for proteins and peptides; this stabilization is achieved by the interaction between the proteins or peptides and the hydrophilic regions and hydrophobic cavities in the cyclodextrins [8].

In recent decades, selective and specific photodiagnostics and treatment of malignant tissue has developed into a separate medical field [10, 11], primarily employing the use of photophysical and photodynamic effects for diagnosis. It is also known that various coumarins and coumarin-associated products are the subject of various studies for estimating the potential therapeutic possibilities for cancer treatment [12].

Because of its optimal photophysical properties, coumarin 6, a green-fluorescing member of the virtually water-insoluble 7-aminocoumarins, has already been used as a laser reference substance for some time [1,4]. By means of the complexing between the coumarin 6 and cyclodextrins (especially beta- and gamma-cyclodextrin), green-fluorescing inclusion complexes can be prepared which are readily soluble in water or buffer, so that even aqueous solutions can be prepared with a relatively high concentration of coumarin 6. With the inclusion complexes, aqueous coumarin 6 solutions can be prepared which have an up to 1,000 times higher concentration of coumarin 6 in comparison to aqueous solutions of pure coumarin 6. In the same way, by the formation of inclusion complexes, aqueous solutions of other coumarins with a relatively high coumarin concentration can be prepared as well.

The object of the present invention is to furnish a method and means which make it possible to mark well-differentiated tumor cells, optionally directly in body fluids, and as a consequence to analyze them both qualitatively and quantitatively.

This object is attained according to the invention by a method including the following steps:

a) generating a dye complex from an aminocoumarin and cyclodextrin;
b) mixing the tumor cells with the dye complex prepared in step a);
c) incubating the cells with the dye complex prepared in step a);
d) analyzing the tumor cells by means of fluorescence microscopy and/or fluorescence spectrometric analysis.

The advantages of the method of the invention are among others that by the use of water-soluble complexes of aminocoumarin and cyclodextrins, the use of organic solvents that irritate cells or tissue can be avoided, and a physiologically favorable form for diagnosis of tumor cells becomes possible.

In the method of the invention, the dye complexes of aminocoumarins and cyclodextrins serve the purpose of ex-vivo marking of cells that are output into the ambient fluid as a result of a lesion. The selective enrichment of the dye complex is due to various morphological differences between tumor cells and healthy cells, since both benign and malignant changes are associated with an altered cell metabolism.

Since well-differentiated tumor cells are already fluorescence-positive, with the method of the invention such cells in the presence of healthy cells, or cell components, can be specifically marked and as a consequence detected, in particular even in body fluids. Thus the method of the invention is capable of unambiguously marking tumor cells, and blood cells such as erythrocytes that are present in the specimens produce no significant background signals or interfering signals. By the method of the invention, the marking and/or detection of tumor cells can be done in separated (isolated), concentrated, or suspended tumor cells, but also directly in tumor cells in the body fluids at various pH values. The pH values can range from pH 2 to pH 11, preferably from pH 6.5 to pH 8.5, as for instance in the urine, and also preferably from pH 6.5 to pH 7.5, as for instance in the blood. It is also possible to analyze tumor cells in sputum and other body fluids.

Since in the method of the invention virtually no interactions with healthy cells, cell components or the suspension solution (water, buffer, urine, etc.) occur, it is also unnecessary, because of this high specificity to tumor cells, to remove the dye complex for the detection/evaluation.

Once the dye complex has bonded to the tumor cells, the thus-marked cells can be detected, distinguished and analyzed by means of fluorescence microscopy and/or fluorescence spectrometry.

In cell-biological study series, it has been possible to show that the method of the invention preferentially marks malignant or pathologically remarkable cells, and distinguishable deposition patterns of the dye complex also occur at different stages of differentiation of the same tumor cells or different tumor cells.

Thus it has been possible to show that in urothelial tumor cells, certain “microvilli”-like structures on the cell membrane, which manifest themselves in bubble-like structures on the exterior of the cells, become preferentially dyed. In contrast to the urothelial cells, in tumorigenic fibroblasts, only an intracellar accumulation occurs, without any strong interaction with the cell membrane.

It can moreover be said that the distribution pattern of the dye complex varies at different stages of differentiation of the tumor cells. Thus in cells at differentiation stage 2, a point-wise accumulation of the dye occurs in the interior of the cell; this is not so pronounced in stage-4 cells.

This is a further advantage of the method of the invention, because when the aminocoumarin dye is used by itself, that is, in the absence of cyclodextrins, in an aqueous/ethanol solution, only slight coloration and no pronounced differences occur in the distribution pattern of the dye-marked tumor cells, making it impossible to distinguish between the various stages of differentiation.

It has been found that with cyclodextrins, a number of aminocoumarins form dye complexes that can be used according to the invention.

The aminocoumarins that can be used for the method of the invention include coumarin 6 (3(-2-benzothiazolyl)-7-(diethylamino)coumarin, coumarin 30 (also called coumarin 515; 7-(diethylamino)-3-(1-methyl-1H-benzo[d]imidazol-2-yl)-2H-chromen-2-on)), coumarin 35 (7-diethylamino-4-trifluoromethylcoumarin), coumarin 47 (7-diethylamino-4-methylchromen-2-on), coumarin 102 (also called coumarin 480; 2,3,6,7-tetrahydro-9-methyl-1H,5H-quinolizino(9,1-gh)coumarin or 8-methyl-2,3,5,6-1H,4H-tetrahydroquinolizino[9,9a,1-gh]coumarin), coumarin 120 (7-amino-4-methylcoumarin), coumarin 138 (8-dimethylamino)cyclopenta[c]chromen-4(3aH)-on), coumarin 151 (7-amino-4-trifluoromethylcoumarin), coumarin 152 (7-dimethylamino-4-trifluoromethylcoumarin), coumarin 153 (2,3,6,7-tetrahydro-9-trifluoromethyl-1H,5H,11H-(1)benzopyranol[6,7,8-ij]quinolizin-11-on), and coumarin 500 (7-(ethylamino-4-(trifluoromethyl)-2H-chromen-2-on).

In an especially preferred embodiment of the invention, coumarin 6, that is, 3(-2-benzothiazolyl)-7-(diethylamino)coumarin, is used.

Cyclodextrins that are especially well-suited for use in the present invention are alpha-cyclodextrin with 6 alpha-1,4-bonded alpha-D-glycopyranose units; beta-cyclodextrin with 7 alpha-1,4-bonded alpha-D-glycopyranose units; and gamma-cyclodextrins with 8 or more alpha-1,4-bonded alpha-D-glycopyranose units. Preferably, beta- and/or gamma-cyclodextrin is used for forming the dye complexes.

The reaction to the dye complex takes place preferably in an aqueous or buffered solution at pH 2 to pH 11, preferably pH 5 to pH 8.5, more preferably pH 6.5 to pH 7.5, in that an alcoholic solution of the aminocoumarin and an aqueous solution of the cyclodextrin are mixed in a molar ratio of aminocoumarin to cyclodextrin of 1:2,000 to 1:20,000 and the water-soluble dye complex is formed at ambient temperature.

In a preferred embodiment of the present invention, the molar ratio of aminocoumarin to cyclodextrin is 1:10,000.

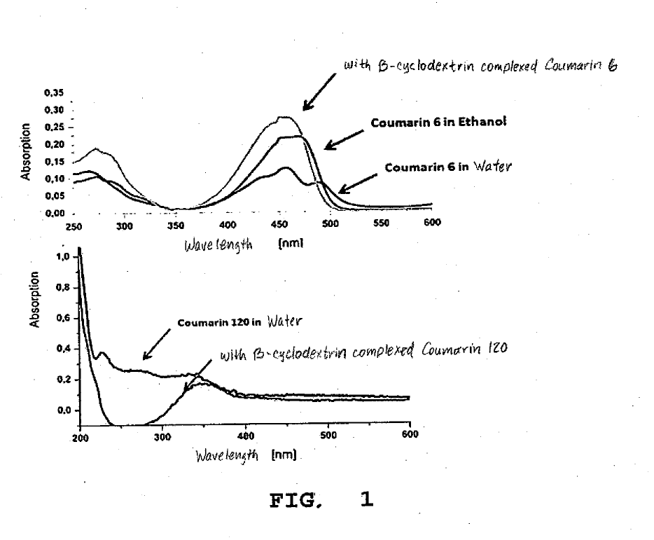

As the most important chemical characterization of the degree of complexing, one can observe the occurrence of additional absorption peaks, which in the case of coumarin 6 (FIG. 1, top) are red-shifted and in the case of coumarin 120 (FIG. 1, bottom) are blue-shifted, as long as the substance is in an aqueous environment. If the coumarins are in an ethanol environment or are kept in an aqueous environment by means of cyclodextrin, these additional peaks do not occur, because the substances are for the most part present as monomers, and not for the most part as aggregates as is normally the case in an aqueous environment. This effect can be explained primarily by hydrophobic interactions or aromatic “stacking”. If the substance is dissolved in a suitable solvent or complexed by means of cyclodextrin, then the hydrophobic/aromatic interactions are reduced to a minimum.

It has been found that the color specificity of the dye complexes decreases after several days, if the dye complexes are stored in an aqueous or buffered solution. After a week, for example, an altered color pattern of the tumor cells occurs, if the dye complex solution is stored in the liquid phase at 4° C. In a preferred embodiment of the present invention, unless an immediate use is planned, the dye complexes are used in the form of lyophilisates. As it has been possible to ascertain, the lyophilisate of the dye complex itself has an unchanged color pattern even after being stored for two weeks at ambient temperature. The lyophilisate can easily be resolubilized in a salt solution or buffer that contains tumor cells, at pH 2 to pH 11, preferably pH 5 to pH 8.5, and more preferably pH 6.5 to pH 7.5, or preferably in body fluids, such as urine, sputum or blood that contains tumor cells, at pH 2 to pH 11, preferably pH 5 to pH 8.5, and more preferably pH 6.5 to pH 7.5. In a further preferred embodiment of the invention, the lyophilisate can also be applied to a solid substrate.

The low concentrations of the dye complex (10 nM coumarin) that are used in the present invention, and the short incubation times of 10 minutes or less, enhance the affinity of the dye complex for tumor cells, and here a different cholesterol content between the normal and the malignant cell appears to play a role. These short incubation times are advantageously attained at from 5° C. to 45° C., preferably at an ambient temperature of from 18° C. to 22° C., especially preferably at 20° C., or at body temperature of from 35° C. to 39° C., especially preferably at 37° C.

For marking and/or detection by means of the method of the invention, the tumor cells can in separated (isolated), concentrated, or suspended form; preferably, however, the tumor cells are present directly in the body fluids. If desired, a step of sedimentation of the tumor cells can therefore be provided in the method of the invention.

The invention will be described below in further detail in conjunction with FIGS. 1-9 and the appended examples.

FIG. 1 shows the absorption spectra of coumarin 6 (complexed/uncomplexed) and coumarin 120 (complexed/uncomplexed) in various aqueous and ethanol solutions.

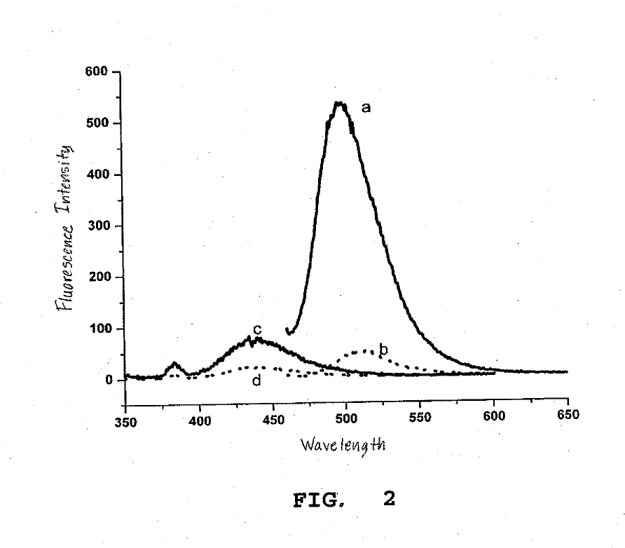

FIG. 2 shows the fluorimetric analysis of stage-4 human urothelial tumor cells in PBS (phosphate buffered saline) at pH 7.4, which were incubated with coumarin-120- and coumarin-6-cyclodextrin dye complex, compared to the background fluorescence of c6 and c12, respectively in beta-cyclodextrin. The greater fluorescence intensity of coumarin 6 (a) by a factor of 6 in comparison to coumarin 120 (c) is clearly apparent, if the cells were incubated with the dye complexes. Nevertheless, an unambiguous fluorescence of the cells marked with coumarin-120-beta-cyclodextrin is apparent. In comparison, the background fluorescence of the coumarin 6 dye complex (b) is around 10% and of the coumarin 120 dye complex (d) is less than 30%.

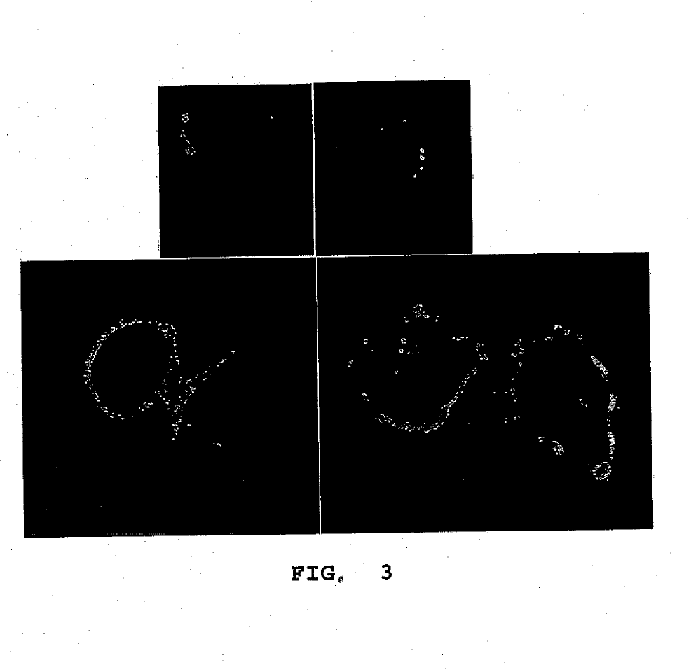

FIG. 3 shows stage-4 human urothelial tumor cells (top left and bottom left) and stage-2 human urothelial tumor cells (top right and bottom right) (all in PBS at pH 7.4), incubated with coumarin-6-beta-cyclodextrin complex (bottom) and coumarin 6 in aqueous/ethanol solution (each at the top). The strong interaction between the urothelial cell membranes and the dye complex can be seen clearly. In addition, a varying intracellular distribution pattern can be seen between the various stages of differentiation. The pattern interaction and distribution of the urothelial cells that have been dyed with the dye complex differs fundamentally from the pattern of intensity of the cells that have been dyed with the pure ethanol/aqueous coumarin 6 solution.

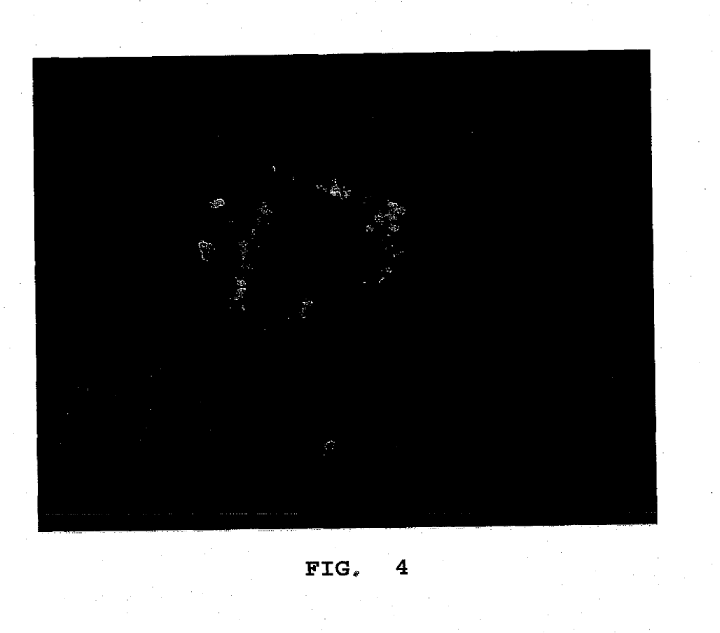

FIG. 4 shows tumor fibroblasts (in PBS at pH 7.4), incubated with coumarin-6-beta-cyclodextrin complex. The weak interaction between the cell membrane and the dye complex can be seen clearly. However, there is an extremely high dye concentration in the interior of the cell.

FIG. 5 shows stage-4 human urothelial tumor cells (in PBS at pH 7.4), incubated with coumarin-6-beta-cyclodextrin complex that have been mixed with human erythrocytes. In transmitted light (on the left), the two types of cell can be clearly distinguished. In the fluorescence image (on the right), only the dyed tumor cells are apparent.

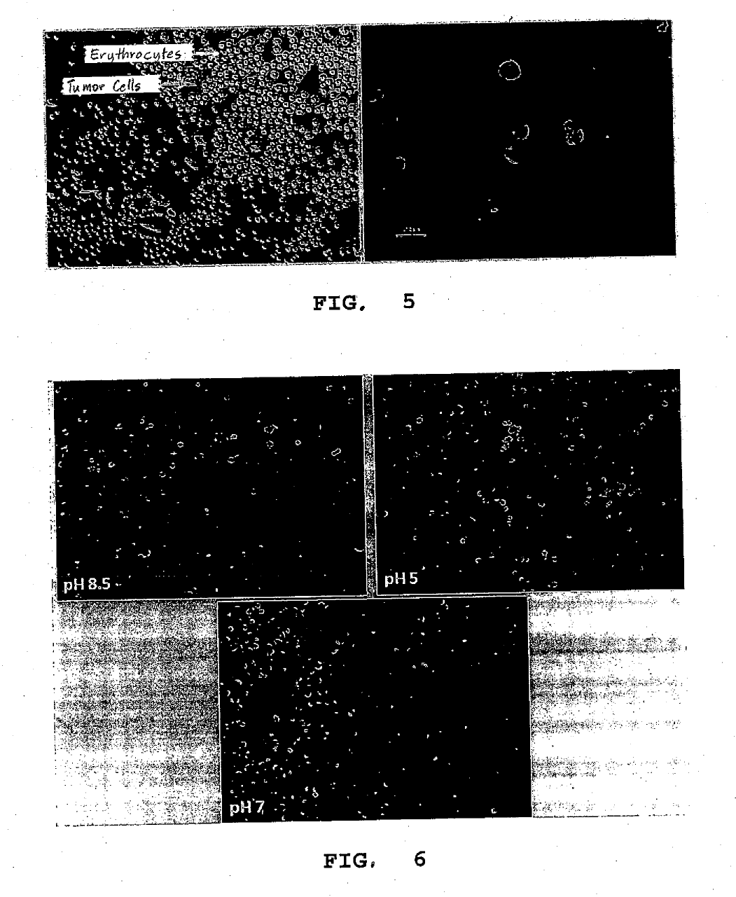

FIG. 6 shows human urothelial tumor cells, incubated with coumarin-6-beta-cyclodextrin complex, in urea solutions at pH values of 5, 7, and 8.5. In all the environments, an identical coloration of the cells is apparent, and there is no significant interaction between the dye complex and the protein found in the urine.

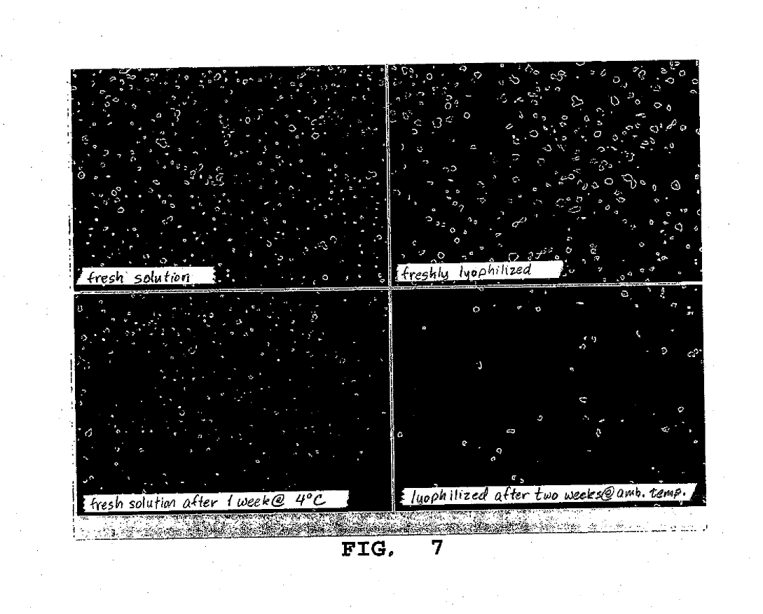

FIG. 7 shows stage-4 human urothelial tumor cells (in PBS at pH 7.4), dyed with a freshly prepared solution of coumarin 6 and beta-cyclodextrin (top/left) and with the same solution after storage a\for 1 week at 4° C. (bottom/left). In comparison, the same cell line is dyed by the addition of freshly lyophilized coumarin-6-beta-cyclodextrin complex to the cell suspension containing tumor cells (top/right) and by the addition of lyophilized coumarin-6-beta-cyclodextrin complex, after storage for 2 weeks at ambient temperature, to the cell suspension containing tumor cells (bottom/right).

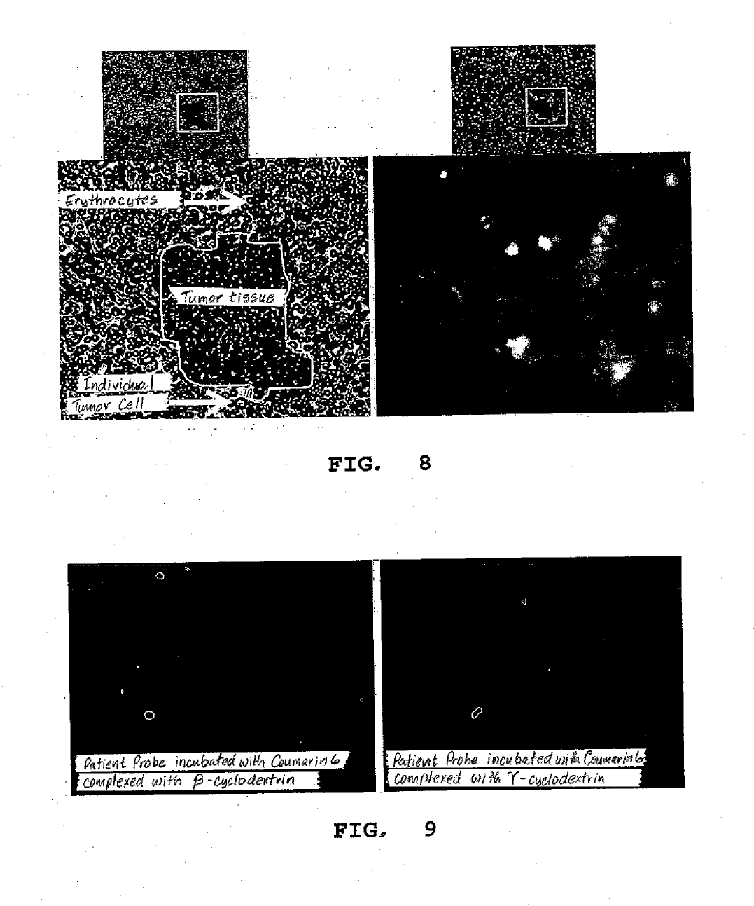

FIG. 8 shows a patient specimen (kidney lavage) incubated with coumarin-6-beta-cyclodextrin complex. The difference in intensity between tumor cell (light) or tumor tissue and erythrocytes is clearly apparent.

FIG. 9 shows a patient specimen (kidney lavage) incubated with coumarin-6-beta-cyclodextrin complex (left) and coumarin-6-gamma-cyclodextrin complex (right). It is clear that there is no difference in the color intensity.

### EXAMPLE 1

### Direct Dyeing with Liquid Dye Solution—Coumarin 6 and Beta-Cyclodextrin

100 mg of beta-cyclodextrin (Sigma-Aldrich, Austria) are dissolved in 10 ml of water in the ultrasonic bath while being heated to 65° C. To 10 ml of this solution, while stirring, 0.5 ml of a 1:1,000 dilution (in ethanol) of 35 mg of 3-(2-benzothiazolyl)-7-(diethylamino)coumarin in 5 ml of ethanol is added, so that a molar ratio of 3-(2-benzothiazolyl)-7-(diethylamino)coumarin to beta-cyclodextrin of 1:10,000 is obtained. The dye complex solution thus obtained, which contains a final concentration of 1 μM of coumarin, is used for marking the tumor cells.

0.01 ml of the fresh dye complex solution is added to 1 ml of a specimen containing tumor cells. After incubation for 10 minutes at ambient temperature, the specimens are examined and evaluated spectroscopically and by fluorescence microscopy.

### EXAMPLE 2

### Direct Dyeing with Liquid Dye Solution—Coumarin 6 and Gamma-Cyclodextrin

110 mg of gamma-cyclodextrin (Sigma-Aldrich, Austria) are dissolved in 10 ml of water at ambient temperature.

To 10 ml of this solution, while stirring, 0.5 ml of a 1:1,000 dilution (in ethanol) of 35 mg of 3-(2-benzothiazolyl)-7-(diethylamino)coumarin in 5 ml of ethanol is added, so that a molar ratio of 3-(2-benzothiazolyl)-7-(diethylamino)coumarin to gamma-cyclodextrin of 1:10,000 is obtained. The dye complex solution thus obtained, which contains a final concentration of 1 μM of coumarin, is used for marking the tumor cells.

0.01 ml of the fresh dye complex solution is added to 1 ml of a specimen containing tumor cells. After incubation for 10 minutes at ambient temperature, the specimens are examined and evaluated spectroscopically and by fluorescence microscopy.

### EXAMPLE 3

### Dyeing of Tumor Cells by Means of Lyophilisate—Coumarin 6 and Beta-Cyclodextrin

100 mg of beta-cyclodextrin (Sigma-Aldrich, Austria) are dissolved in 10 ml of water in the ultrasonic bath while being heated to 65° C.

To 10 ml of this solution, while stirring, 0.5 ml of a 1:1,000 dilution (in ethanol) of 35 mg of 3-(2-benzothiazolyl)-7-(diethylamino)coumarin in 5 ml of ethanol is added, so that a molar ratio of 3-(2-benzothiazolyl)-7-(diethylamino)coumarin to beta-cyclodextrin of 1:10,000 is obtained. The thus-obtained dye complex solution is then transferred to a reaction vessel and lyophilized until dry. The lyophilisate has a final concentration of 1 μM of coumarin.

1 mg of the lyophilisate is added to 10 ml of a specimen containing tumor cells. After incubation for 10 minutes at ambient temperature, the specimens are examined and evaluated spectroscopically and by fluorescence microscopy.

### EXAMPLE 4

### Dyeing of Tumor Cells by Means of Lyophilisate—Coumarin 6 and Gamma-Cyclodextrin

100 mg of gamma-cyclodextrin (Sigma-Aldrich, Austria) are dissolved in 10 ml of water in the ultrasonic bath while being heated to 65° C.

To 10 ml of this solution, while stirring, 0.5 ml of a 1:1,000 dilution (in ethanol) of 35 mg of 3-(2-benzothiazolyl)-7-(diethylamino)coumarin in 5 ml of ethanol is added, so that a molar ratio of 3-(2-benzothiazolyl)-7-(diethylamino)coumarin to gamma-cyclodextrin 1:10,000 is obtained. The thus-obtained dye complex solution is then transferred to a reaction vessel and lyophilized until dry. The lyophilisate has a final concentration of 1 μM of coumarin.

1.1 mg of the lyophilisate is added to 10 ml of a specimen containing tumor cells. After incubation for 10 minutes at ambient temperature, the specimens are examined and evaluated spectroscopically and by fluorescence microscopy.

### EXAMPLE 5

### Dyeing of Tumor Cells by Means of Lyophilisate—Coumarin 120 and Beta-Cyclodextrin

100 mg of beta-cyclodextrin (Sigma-Aldrich, Austria) are dissolved in 10 ml of water in the ultrasonic bath while being heated to 65° C.

To 10 ml of this solution, while stirring, 0.5 ml of a 1:1,000 dilution (in ethanol) of 17.5 mg of 3-(2-benzothiazolyl)-7-(diethylamino)coumarin in 5 ml of ethanol is added, so that a molar ratio of 7-amino-4-methylcoumarin to beta-cyclodextrin of 1:10,000 is obtained. The thus-obtained dye complex solution is then transferred to a reaction vessel and lyophilized until dry. The lyophilisate has a final concentration of 1 μM of coumarin.

1 mg of the lyophilisate is added to 10 ml of a specimen containing tumor cells. After incubation for 10 minutes at ambient temperature, the specimens are examined and evaluated spectroscopically and by fluorescence microscopy.

### LITERATURE

1. Creaven, P. J., D. Parke, and R. T. Williams, A Spectrofluorimetric Study of 7-Hydroxylation of Coumarin by Liver Microsomes, Biochemical Journal, 1965. 96(2): p. 390-&.
2. Kim, H. S. and I. W. Wainer, Rapid analysis of the interactions between drugs and human serum albumin (HSA) using high-performance affinity chromatography (HPAC). Journal of Chromatography B—Analytical Technologies in the Biomedical and Life Sciences, 2008. 870(1): p. 22-26.
3. Liu, X. H., et al., Spectroscopic studies on binding of 1-phenyl-3-(coumarin-6-yl)sulfonylurea to bovine serum albumin. Journal of Photochemistry and Photobiology B—Biology, 2008. 92(2): p. 98-102.
4. Pelkonen, O. and H. Raunio, Metabolic activation of toxins: Tissue-specific expression and metabolism in target organs. Environmental Health Perspectives, 1997. 105: p. 767-774.
5. Prabu, P., et al., In vitro evaluation of poly(caprolactone) grafted dextran (PGD) nanoparticles with cancer cell. Journal of Materials Science—Materials in Medicine, 2008. 19(5): p. 2157-2163.
6. Kovalenko, S. N., Stepanian, S. G., Chuev, V. P., Asimov, M. M., Nikitchenko, V. M., and Chemykh, V. P., Modelling of Formation Processes of Inclusion Complexes of Coumarin Laser Dyes and β-Cyclodextrin by the MM2 Force Field Method. I. 7-Amino-4-methylcoumarins Mol. Eng., 1992. 2: p. 153-163.
7. Velic, D., Knapp, M. and Köhler, G., Supramolecular inclusion complexes between a coumarin dye and β-cyclodextrin, and attachment kinetics of thiolated β-cyclodextrin to gold surface. J. Mol. Struct., 2001. 598: p. 49-56.
8. Vyas, A., S. Saraf, and S. Saraf, Cyclodextrin based novel drug delivery systems. Journal of Inclusion Phenomena and Macrocyclic Chemistry, 2008. 62(1-2): p. 23-42.
9. Hirayama, F. and K. Uekama, Cyclodextrin-based controlled drug release system. Advanced Drug Delivery Reviews, 1999. 36(1): p. 125-141.
10. Dougherty, T. J. and S. L. Marcus, Photodynamic Therapy. European Journal of Cancer, 1992. 28A (10): p. 1734-1742.
11. Vanhillegersberg, R., W. J. Kort, and J. H. P. Wilson, Current Status of Photodynamic Therapy in Oncology. Drugs, 1994. 48(4): p. 510-527.
12. Lacy, A. a. O. K., R. Studies on Coumarins and Coumarin-Related Compounds to Determine their Therapeutic Role in the Treatment of Cancer. Current Pharmaceutical Design, 2004. 10(30): p. 3797-811.
13. Frank, S. G., Inclusion Compounds. J. Pharm. Sci., 1978. 64: p. 1585-604.
14. Fery-Forgues, S., R. El-Ayoubi, and J. Lamère, F., Fluorescent Microcrystals Obtained from Coumarin 6 using the Reprecipitation Method. J Fluorescence, 2008. 18: p. 619-624.
15. Hicks, R. M. and J. S. J. Wakefield, Membrane Changes during Urothelial Hyperplasia and Neoplasia. Cancer Research, 1976. 36(7): p. 2502-2507.

1. A method for qualitative and/or quantitative analysis of tumor cells, including the following steps:
a) generating a dye complex from an aminocoumarin and cyclodextrin;
b) mixing the tumor cells with the dye complex prepared in step a);
c) incubating the cells with the dye complex prepared in step a);
d) analyzing the tumor cells by means of fluorescence microscopy and/or fluorescence spectrometric analysis.

2. The method as defined by claim 1, wherein the aminocoumarin is selected from the group including coumarin 6, coumarin 30 (also coumarin 515), coumarin 35, coumarin 47, coumarin 102 (also coumarin 480), coumarin 120, coumarin 138, coumarin 151, coumarin 152, coumarin 153, coumarin 500.

3. The method as defined by claim 1 wherein the cyclodextrin is selected from the group including alpha-cyclodextrin, beta-cyclodextrin, or gamma-cyclodextrin.

4. The method as defined by claim 1, wherein the molar ratio of aminocoumarin and cyclodextrin ranges from 1:2,000.

5. The method as defined by claim 1, wherein the dye complex is used in dissolved and/or lyophilized form.

6. The method as defined by claim 1, wherein method step c) takes place in a temperature range of 5° C. to 45° C. or at body temperature of 35° C. to 39° C.

7. The method as defined by claim 1, wherein the tumor cells are present in body fluids.

8. The method as defined by claim 1, wherein the tumor cells, after method step c) and before the fluorescence microscopic analysis and/or fluorescence spectrometric analysis, are subjected to a separation step.

9. Use of a dye complex comprising aminocoumarin and cyclodextrin for marking tumor cells.

10. The use as defined by claim 9, characterized in that the aminocoumarin is selected from the group including coumarin 6, coumarin 47, coumarin 120, coumarin 138, coumarin 151, coumarin 152, coumarin 153, and is preferably coumarin 6.

11. The use as defined by claim 9 or 10, characterized in that the group including alpha-cyclodextrin, beta-cyclodextrin, or gamma-cyclodextrin, and is preferably beta- and/or gamma-cyclodextrin.

12. The use as defined by one of claims 9-11, characterized in that the molar ratio of aminocoumarin and cyclodextrin ranges from 1:2,000 to 1:20,000 and is preferably 1:10,000.

13. The method as defined by claim 1, characterized in that the aminocoumarin is selected from the group including coumarin 6, coumarin 30 (also coumarin 515), coumarin 35, coumarin 47, coumarin 102 (also coumarin 480), coumarin 120, coumarin 138, coumarin 151, coumarin 152, coumarin 153, coumarin 500, and is preferably coumarin 6.

14. The method as defined by claim 1 or 2, characterized in that the cyclodextrin is selected from the group including alpha-cyclodextrin, beta-cyclodextrin, or gamma-cyclodextrin, and is preferably beta- and/or gamma-cyclodextrin.

15. The method as defined by one of claims 1-3, characterized in that the molar ratio of aminocoumarin and cyclodextrin ranges from 1:2,000 to 1:20,000 and is preferably 1:10,000.

16. The method as defined by one of claims 1-5, characterized in that method step c) takes place in a temperature range of 5° C. to 45° C., preferably at ambient temperature of from 18° C. to 22° C., especially preferably at 20° C., or at body temperature of 35° C. to 39° C., especially preferably at 37° C.
